(19) United States
(12) Patent Application Publication
(10) Pub. No.: US 2006/0128667 A1
(43) Pub. Date: Jun. 15, 2006

(54) Hydroxyphosphonates and phosphonophosphates as apolipoprotein e modulators

(76) Inventors: Imber Flores Montes, Le Lignon (CH); Hieu Trung Phan, Tannay (CH); Lan Mong Nguyen, Nyon (CH); Emanuele Burattini, Geneva (CH); Eric Joseph Neisor, Nyon (CH); Anne Perez, Versoix (CH); Jean Luc Thuillard, Saint Cergue (CH); Yves Guvon-Gellin, Annemasse (FR); Craig Leigh Bentzen, Bogi-Bossey (CH)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/513,553

(22) PCT Filed: May 9, 2003
(86) PCT No.: PCT/US03/14728

§ 371 Date: Oct. 21, 2005

Related U.S. Application Data
(60) Provisional application No. 60/379,900, filed on May 11, 2002.

(51) Int. Cl. A61K 31/675; C07F 9/547

(52) U.S. Cl. 514/80; 546/22; 548/415; 514/89

(57) ABSTRACT

The present invention relates to the novel hydroxyphosphonates and phosphonophosphates and the methods of their use to modulate apolipoprotein E levels and the use of such compounds in therapy, including cardiovascular and neurological disease states.

### FIELD OF INVENTION

The present invention relates to hydroxyphosphonate and phosphonophosphate compounds, the processes for their preparation, pharmaceutical compositions containing them and their use in therapy, in particular for modulating (increasing or decreasing) apolipoprotein E in plasma and in tissues.

### BACKGROUND OF THE INVENTION

Apolipoprotein E (apoE) is a polymorphic, multifunctional protein synthesized by several cell types and tissues, including liver, kidney, skin, adipose tissue, macrophages and brain. The wide distribution of apoE is associated with the maintenance of key cellular functions such as intracellular cholesterol trafficking, cholesterol distribution between cells, and tissue reparation.

The amino acid sequence of the apoE protein is well conserved throughout species. ApoE can be viewed as a regulator of cholesterol homeostasis in tissues such as the central nervous system (CNS) and peripheral nervous system (PNS) and the arterial wall (cell-cell) or between tissues via the circulating plasma lipoproteins (tissue-tissue).

The major role of plasma apoE containing lipoproteins is to transfer lipids (cholesterol) from peripheral tissues to the liver and to remove excess cholesterol from peripheral tissues via the reverse cholesterol transport system. Dysregulation of this mechanism leads to excess cholesterol deposition in peripheral tissues such as arteries (arterosclerosis) and skin (xanthomas and xanthelasmas). ApoE has also been shown to have a direct effect on lymphocyte proliferation and thus has an immunomodulatory role.

ApoE is the only lipoprotein synthesized in the brain and has a key role in cholesterol transport between cells of the CNS. Local secretion of apoE by cells such as macrophages or macrophage-derived cells is essential for the uptake of excess tissue cholesterol and the provision of cholesterol for specific needs such as nerve repair and remyelinisation.

Up to the present time, compounds affecting Apo E production in vitro and in vivo have not been extensively investigated. Only hormone-like estrogens and corticoids have been shown to change ApoE levels under various experimental conditions (Srivastava et al., 1997; Stone et al., 1997).

There is currently a need for compounds that modulate apoE synthesis and secretion, such compounds having application in the treatment of diseases such as atherosclerosis, excess lipid deposition in peripheral tissues such as skin (xanthomas), stroke, memory loss, optic nerve and retinal pathologies (i.e., macular degeneration, retinitis pigmentosa), repair of traumatic damage of the central nervous system (brain tissue), repair of traumatic damage of the peripheral nervous system (i.e., nerve section compression or crush), prevention of the degenerative process due to aging (i.e., Alzheimer's disease), prevention of degenerative neuropathies occurring in diseases such as diabetic neuropathies and multiple sclerosis, autoimmune diseases and activation of the innate immune system.

### SUMMARY OF THE INVENTION

The Applicants have now found that certain hydroxyphosphonates and phosphonophosphates modulate (increase or decrease) the production of apoE. One aspect of the present invention are phosphonate derivatives of formula (I): 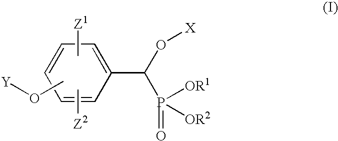
wherein:
Y is hydrogen, aryl, C₂-C₆ alkyl, PO(OR⁵OR⁶), R³R⁴N(CH₂)ₘ— or A-L;
A is aryl or heterocycle;
L is —(CH₂)ᵣ—, —(CH₂)ₚO(CH₂)_{q}—, —(CH₂)ₚN(R⁷)(CH₂)_{q}— or —(CH₂)ₚNHCO(CH₂)_{q}— wherein
R⁷ is hydrogen, C₁-C₄ alkyl, aryl or C₁-C₃ cyanoalkyl;
m, p, q and r are independently an integer from 0 to 6;
X is hydrogen or PO(OR⁵OR⁶);
R¹, R² and R⁵, R⁶ are independently hydrogen or C₁-C₆ alkyl;
R³ and R⁴ are independently hydrogen or C₁-C₄ alkyl;
and Z¹ and Z² are independently hydrogen, C₁-C₄ alkyl, or C₁-C₄ alkoxy;
with the proviso that when X is hydrogen, then Y—O—, Z¹ and Z² are not all independently hydroxy, hydrogen, alkoxy or alkyl;

The invention also encompasses pharmaceutically acceptable salts of the compounds of formula (I).

In various embodiments, Z¹ and Z² are hydrogen and Y is hydrogen, optionally substituted aryl or diethoxyphosphinyl. A may suitable be pyridin-2-yl, 5-methyl-pyridin-2-yl, pyridin-3-yl, N-phthalimido, phenyl-4-yl or p-cyanophenyl. In some embodiments R¹, R² and R⁵ are independently methyl or ethyl. In various embodiments the phosphonate derivative is diethyl 1-hydroxy-1-{4-[3-N-phthalimido-propoxy]-phenyl}-methylphosphonate, diethyl 1-(diethoxy-phosphinyloxy)-1-[3-(pyridin-3-yl-methoxy)-phenyl]-methylphosphonate, diethyl I-(diethoxy-phosphinyloxy)-1-[3-(2-pyridin-2-yl-ethoxy)-phenyl]-methylphosphonate, diethyl 1-(diethoxy-phosphinyloxy)-1-[4-(2-pyridin-2-yl-ethoxy)-phenyl]-methylphosphonate, diethyl 1-hydroxy-1-{4-[2-(methyl-pyridin-2-yl-amino)-ethoxy]-phenyl}-methylphosphonate, diethyl 1-(diethoxy-phosphinyloxy)-1-[3-(3-N-phthalimido-propoxy)-phenyl]-methylphosphonate, or diethyl 1-(diethoxy-phosphinyloxy)-1-[3-(5-N-phthalimido-pentoxy)-phenyl]-methylphosphonate. The invention also provides for pharmaceutical compositions of the forgoing phosphonate derivatives, comprising a phosphonate derivative of formula (I) and a pharmaceutically acceptable carrier.

Other aspect of the invention provides for methods of modulating the production of apoE by an apoE producing cell, comprising contacting said apoE producing cell with an effective amount of a compound of formula (I) and modulating the levels of ApoE in a patient in need of such treatment, comprising administration of an effective amount of a compound of formula (1). In some embodiments, the level of apoE is increased and the patient may be suffering from atherosclerosis, Alzheimer's disease, macular degeneration, retinitis pigmentosa, stroke, degenerative neuropathy, xanthoma or xanthelasma. Increasing apoE levels may provide methods for elevating high density cholesterol, preventing and/or treating atherosclerosis, macular degeneration, retinitis pigmentosa, stroke or degenerative neuropathy. Degenerative neuropathy may be associated with diabetic neuropathy or multiple sclerosis. In other embodiments, apoE levels are decreased by administration to a patient of an effective amount of a compound of formula (I). The patient may express apoE4, apoE Leiden or a non-functional mutant form of apoE. The patient may be suffering from atherosclerosis or Alzheimer's disease.

A further aspect of the invention, provides for a method for the prevention and/or treatment of Alzheimer's disease or dementia comprising administration to a patient an effective amount of a compound of formula (I). The patient may be heterozygous or homozygous for apoE2 and/or apoE3 and the administration of an effective amount of a compound of formula (I) increases apoE levels. Alternatively, the patient may be heterozygous or homozygous for apoE4 and the administration of an effective amount of a compound of formula (I) decreases apoE levels.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Schematic summary of preparation of hydroxyphosphonates of formula (Ia) and phosthonophosphates of formula (Ib). Substituents Y, Z¹, Z², R¹, R², R⁵ and R⁶ are as described in Detailed Description of the Invention.

FIG. 2—Schematic summary of alternative preparation of phosphonophosphates of formula (Ib). Substituents Y, Z¹, Z², R¹, R², R¹ and R⁶ are as described in Detailed Description of the Invention.

### DETAILED DESCRIPTION OF THE INVENTION

I. Hydroxyphosphonate and Phosphonophosphate Compounds

The present invention relates to novel hydroxyphosphonate and phosphonophosphate compounds of general formula (I) that modulate apoE levels and are useful as agents for the treatment of a number of disorders including cardiovascular and neurological disease states.

As used herein, the term “aryl” refers to an unsaturated aromatic carbocyclic group of from 6 to 14 carbon atoms having a single ring (e.g., phenyl) or multiple condensed (fused) rings (e.g., naphthyl or anthryl). Suitable aryls include phenyl, naphthyl and the like. Unless otherwise constrained by the definition for the aryl substituent, such aryl groups can optionally be substituted with from 1 to 5 substituents and preferably 1 to 3 substituents selected from the group consisting of hydroxy, alkyl (e.g., methyl, ethyl, n-propyl, isopropyl, sec-butyl, or tert-butyl), alkoxy (e.g., methoxy, ethoxy, propoxy, tert-butoxy), cyano, amidino, cyanoalkyl (e.g., cyanomethyl, cyanoethyl and cyanopropyl), aryl (e.g., phenyl), halo (e.g., I, Br, Cl, F) or nitro.

As used herein, the term “heterocycle” refers to aromatic and non-aromatic heterocyclic groups and refers to a single ring or fused rings containing up to four heteroatoms in at least one ring, each of which is selected from oxygen, nitrogen and sulphur, which single or fused ring may be unsubstituted or substituted. Each ring suitably has from 4 to 7, preferably 5 or 6 ring atoms. Representative examples of heterocyclic groups include pyridin-2-yl, 5-methyl-pyridin-2-yl, pyrridin-3-yl, naphthalimido, phthalimido, succinimido, piperidino, pyrrolidino and morpholino.

Pharmaceutically acceptable salts for use in the present invention include those described by Berge et al., 1997, herein incorporated by reference. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

Since the compounds of the present invention, in particular compounds of formula (I), are intended for use in pharmaceutical compositions, it will be understood that they are each provided in substantially pure form, for example at least 50% pure, more suitably at least 75% pure, preferably at least 95% pure, and more preferably at least 99% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are recrystallised from organic solvents, solvent of crystallisation may be present in the crystalline product. This invention includes within its scope such solvates. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

II. Applications of ApoE Modulators

A. ApoE in Atherosclerosis

As a component of all lipoprotein fractions, apoE plays a important role in cholesterol homeostasis, by mediating their interaction with receptors such as the apoB, low-density lipoprotein (LDL) and other specific receptors. The important role of apoE in cardiovascular diseases is demonstrated by the apoE knock-out mouse model, where the animals rapidly develop hypercholesterolemia and atherosclerosis with pathological features similar to human atherosclerosis (Plump, 1997). In addition, the absence of a functional apoE in humans is associated with abnormally high plasma levels of cholesterol and triglycerides and the rapid development of atherosclerosis, notwithstanding a low fat diet (Richard et al., 1995). In the knock-out mouse model, these changes are prevented by infusion of apoE, transplantation of macrophage producing apoE, or gene therapy by introducing the human apoE gene into apoE knock out mice (Linton et al., 1995). These results indicate a direct beneficial role for apoE and, consequently, a utility for compounds that increase the apoE levels. The hydroxyphosphonate and phosphonophosphate compounds of the present invention that increase apoE plasma levels will decrease plasma atherogenic lipoproteins (VLDL, IDL and LDL) by increasing their uptake by the liver. Increasing apoE in HDL will increase the removal of cholesterol from loaded tissues (atherosclerotic arteries) by the reverse cholesterol transport mechanism.

In contrast, hyperlipidemic patients susceptible of developing atherosclerosis due to the expression of a mutated form of apoE, such as apoE Leiden or other variants, should benefit from the treatment with the compounds that decrease apoE production (van Vlijmen et al., 1998; Richard, 1995). Thus, hydroxyphosphonate and phosphonophosphate compounds of the present invention that decrease the production of apoE are useful in the prevention and/or treatment of pathological cardiovascular conditions secondary to the presence of non-functional, variants or mutant forms of the apoE molecule.

B. ApoE in the Central Nervous System (CNS)

ApoE also plays a critical role in the CNS. In the brain apoE is synthesized and secreted by astrocytes, its principal role being cholesterol transport between cells. ApoE is considered to redistribute lipids and to participate in the cholesterol homeostasis of the brain.

ApoE is linked to the neuropathological lesions characteristic of Alzheimer's disease. One isoform, ApoE4, is strongly associated with the age of onset of the disease (Poirier, 1994; Rubinsztein, 1995), while another isoform, apoE3, is believed to help maintain healthy microtubules. The increase in both apoE mRNA and the number of astrocytes in the brains of Alzheimer's patients, indicates that increased apoE represents an astrocyte repair-mechanism to ameliorate the damage within the nervous cells. Memory deficit, defective repair of brain injury and deposition of the Alzheimer's associated β-amyloid variant APP^{V717F} have been demonstrated in the absence of the apoE gene, i.e., apoE knock out mice (Oitzl et al., 1997; Laskowitz et al., 1997; Walker et al., 1997).

Thus, there is a benefit to increasing apoE production in patients bearing the E2 and E3 isoforms of apoE in regard to the occurrence of Alzheimer's or other spontaneous or traumatic neurological diseases. The hydroxyphosphonate and phosphonophosphate of the present invention that increase apoE in the brain will prevent the deposition of plaques associated with Alzheimer's disease and increase the repair mechanism of brain injuries due to mechanical traumas or strokes. Through the increase of neurite extension synaptic sprouting the overall brain activity (e.g., memory) should improve.

Conversely, patients at risk of or suffering from Alzheimer's or spontaneous or traumatic neurological diseases who overexpress the pathological isoforms of apoE, such as apoE4, should benefit from the treatment with a compound that decreases apoE. Thus, hydroxyphosphonate and phosphonophosphate compounds of the present invention that decrease the production of apoE are useful in the prevention and/or treatment of the symptomatic and neuropathological cardiovascular conditions characteristic of Alzheimer's or other spontaneous or traumatic neurological diseases that are caused or exacerbated by non-functional, variants or mutant forms of the apoE.

C. ApoE in the Peripheral Nervous System (PNS)

The important role of apoE in nerve regeneration in the PNS is demonstrated by the observation that apoE synthesis is dramatically induced when nerves are injured (Poirier, 1994). The maintenance and/or repair of the myelin sheets involves the participation of apoE secreted by support cells such as glial and Schwann cells. Both apoE synthesis and concentration were found to be abnormally low in degenerative diseases of nervous tissues such as in multiple sclerosis (Gaillard, 1996). ApoE is also considered to stabilize the cytoskeleton apparatus and support neurite elongation, thus having a major effect on the development and remodelling following injury of the nervous system occurring late in life. Thus, the compounds of the present invention that increase apoE will support and increase the speed of the healing process of traumatised nerves (nerve section, crush etc) and the prevention and/or healing of degenerative nerves (e.g., multiple sclerosis).

D. ApoE as Modulators of the Immune System

ApoE affects the immune system by acting on lymphocyte proliferation. Furthermore apoE knock out mice are highly sensitive to bacterial infection due to a defect in the innate immune system, suggesting that increasing apoE production should augment the immune response (Roselaar & Daugherty, 1998). Increasing apoE production by utilization of compounds of the present invention should ameliorate the immune response in patients in need thereof.

E. Skin Lipid Metabolism Disorders

Lipid homeostasis is well controlled in epithelial cells such as keratinocytes, wherein exported lipids are important for corneocyte adhesion and for forming the cutaneous barrier to the external enviroment. Excess cholesterol deposition in skin (xanthomas and xanthelasmas) will be prevented by utilization of hydroxyphosphonate and phosphonophosphate compounds of the present invention that increase the level of cutaneous apoE.

III. Formulations and Administration

The compounds of formula (I) can be administered by any of a variety of routes. Thus, for example, they can be administered orally, or by delivery across another mucosal surface (for example across the nasal, buccal, bronchial or rectal mucosa), transdermally, or by injection (for example intradermal, intraperitoneal, intravenous or intramuscular injection).

When the compounds are intended for oral administration, they can be formulated, for example, as tablets, capsules, granules, pills, lozenges, powders, solutions, emulsions, syrups, suspensions, or any other pharmaceutical form suitable for oral administration. Oral dosage forms can, if desired, be coated with one or more release delaying coatings to allow the release of the active compound to be controlled or targeted at a particular part of the enteric tract.

Tablets and other solid or liquid oral dosage forms can be prepared (e.g., in standard fashion) from the compounds of formula (I) and a pharmaceutically acceptable solubilizer, diluent or carrier. Examples of solubilizers, diluents or carriers include sugars such as lactose, starches, cellulose and its derivatives, powdered tracaganth, malt, gelatin, talc, stearic acid, magnesium stearate, calcium sulfate, vegetable oils, polyols such as glycerol, propyleneglycol and polyethyleneglycols, alginic acids and alginates, agar, pyrogen free water, isotonic saline, phosphate buffered solutions, and optionally other pharmaceutical excipients such as disintegrants, lubricants, wetting agents such as sodium lauryl sulfate, coloring agents, flavoring agents and preservatives, etc.

Capsules can be of the hard or soft variety and can contain the active compound in solid, liquid or semisolid form. Typically such capsules are formed from gelatine or an equivalent substance and can be coated or uncoated. If it is desired to delay the release of the active compound until the capsule has passed through the stomach and into the intestine, the capsule can be provided with a pH sensitive coating adapted to dissolve at the pH found in the duodenum or ileum. Examples of such coatings include the Eudragits, the uses of which are well known.

Formulations for injection will usually be made up of the appropriate solubilizers such as detergents which may also include compounds and excipients such as buffering agents to provide an isotonic solution having the correct physiological pH. The injectable solutions are typically pyrogen-free and can be provided in sealed vials or ampoules containing a unit dose of compound.

A unit dosage form of the compounds of the invention typically will contain from 0.1% to 99% by weight of the active substance, more usually from 5% to 75% of the active substance. By way of example, a unit dosage form can contain from 1 mg to 1 g of the compound, more usually from 10 mg to 500 mg, for example between 50 mg and 400 mg, and typically in doses of 100 mg to 200 mg.

The compounds of the invention will be administered in amounts which are effective to provide the desired therapeutic effect. The concentrations necessary to provide the desired therapeutic effect will vary according to among other things the precise nature of the disease, the size, weight and age of the patient and the severity of the disease. The doses administered will preferably be non-toxic to the patient, although in certain circumstances the severity of the disease under treatment may necessitate administering an amount of compound that causes some signs of toxicity.

Typically, the compounds of the invention will be administered in amounts in the range 0.01 mg/kg to 100 mg/kg body weight, more preferably 0.1 mg/kg to 10 mg/kg body weight and particularly 1 mg/kg to 5 mg/kg body weight. For an average human of 70 kg weight, a typical daily dosage of the compounds of the invention would be in the range of 70 mg to 700 mg. Such a dosage can be administered, for example from two to four times daily. Ultimately however, the size of the doses administered and the frequency of administration will be at the discretion and judgment of the physician treating the patient.

For therapeutic use the compounds of the present invention will generally be administered in a standard pharmaceutical composition obtained by admixture with a pharmaceutical carrier selected with regard to the intended route of administration and standard pharmaceutical practice. For example, they may be administered orally in the form of tablets containing such excipients as starch or lactose, or in capsule, ovules or lozenges either alone or in admixture with excipients, or in the form of elixirs or suspensions containing flavoring or coloring agents. They may be injected parenterally, for example, intravenously, intramuscularly or subcutaneously. For parenteral administration, they are best used in the form of a sterile aqueous solution that may contain other substances, for example, enough salts or glucose to make the solution isotonic with blood. The choice of form for administration as well as effective dosages will vary depending, inter alia, on the condition being treated. The choice of mode of administration and dosage is within the skill of the art.

The compounds of formula (I) and their pharmaceutically acceptable salts which are active when given orally can be formulated as liquids, for example syrups, suspensions or emulsions or as solids for example, tablets, capsules and lozenges. A liquid formulation will generally consist of a suspension or solution of the compound or pharmaceutically acceptable is salt in suitable liquid carrier(s) for example, ethanol, glycerine, non-aqueous solvent, for example polyethylene glycol, oils, or water with a suspending agent, preservative, flavoring or coloring agents. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatine capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

A typical suppository formulation comprises a compound of formula (I) or a pharmaceutically acceptable salt thereof which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule.

Each dosage unit for oral administration contains preferably from 1 to 250 mg (and for parenteral administration contains preferably from 0.1 to 25 mg) of a compound of the structure (I) or a pharmaceutically acceptable salt thereof calculated as the free base.

The pharmaceutically acceptable compounds of the invention will normally be administered to a subject in a daily dosage regimen. For an adult patient this may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the compound of the structure (I) or a pharmaceutically acceptable salt thereof calculated as the free base, the compound being administered 1 to 4 times per day.

Disease states which could benefit from increasing plasma and tissue apoE levels include, but are not limited to: atherosclerosis, neurodegenerative disorders such as Alzheimer's disease or dementia. The compounds of this invention modulate apoE and are therefore of value in the treatment of any of these conditions.

Compounds of the present invention may also be of use in preventing and/or treating the above mentioned disease states in combination with anti-hyperlipidaemic, anti-atherosclerotic, anti-diabetic, anti-anginal, anti-inflammatory or anti-hypertension agents. Examples of the above include cholesterol synthesis inhibitors such as statins, for instance atorvastatin, simvastatin, pravastatin, cerivastatin, fluvastatin, lovastatin and ZD 4522 (also referred to as S-4522, Astra Zeneca), antioxidants such as probucol, insulin sensitisers such as a PPAR gamma activator, for instance G1262570 (Glaxo Wellcome) and the glitazone class of compounds such as rosiglitazone (Avandia, SmithKline Beecham), troglitazone and pioglitazone, calcium channel antagonists, and anti-inflammatory drugs such as NSAIDs.

IV. Synthesis of Hydroxyphosphonates (Ia) and Phosphonophosphates (Ib)

The present invention also provides for the preparation of hydroxyphosphonate and phosphonophosphate derivatives of the respective formulas (Ia): 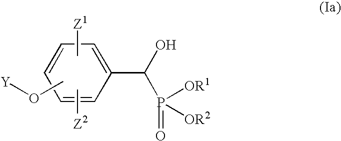
and (Ib): 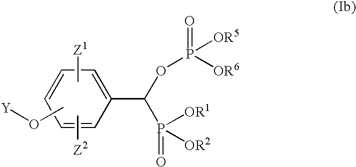
where y, Z¹, Z², R¹R², R⁵ and R⁶ are as described previously.

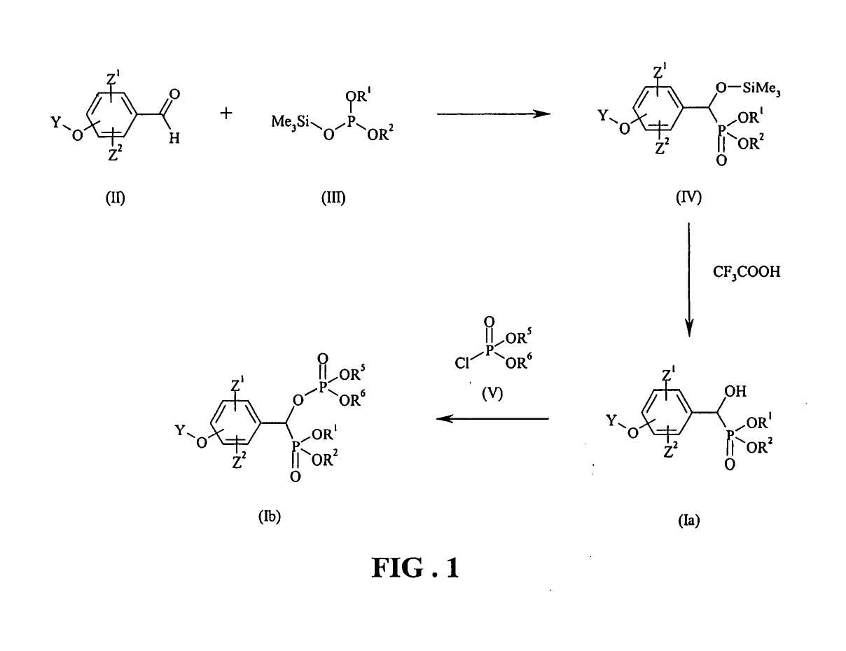

The first method, shown schematically in FIG. 1, comprises heating the aldehyde (II) with a slight excess (1.05 to 1.20 equivalents) of dialkyl trimethylsilyl phosphite (III) to between 100° to 110° C. for a period between 2 to 24 h. After removal of the excess of reagent and side products under vacuum the crude α-trimethylsiloxy phosphonate (IV) is treated with a catalytic amount of trifluoroacetic acid in methanol at room temperature for a period between 6 and 16 h. The crude α-hydroxy phosphonate (Ia) is obtained by evaporation of the volatiles and, if necessary is purified by column chromatography. A solution of α-hydroxy phosphonate (Ia) in 20 ml of THF is treated with a stirred suspension of 1.0 equivalent of NaH in tetrahydrofuran (THF) under nitrogen at a temperature between 0° C. and room temperature. A THF solution of dialkyl chlorophosphate (V) is added to this mixture and allowed to warm to room temperature and stirred for 2-8 h to yield the phosphonophosphate (Ib).

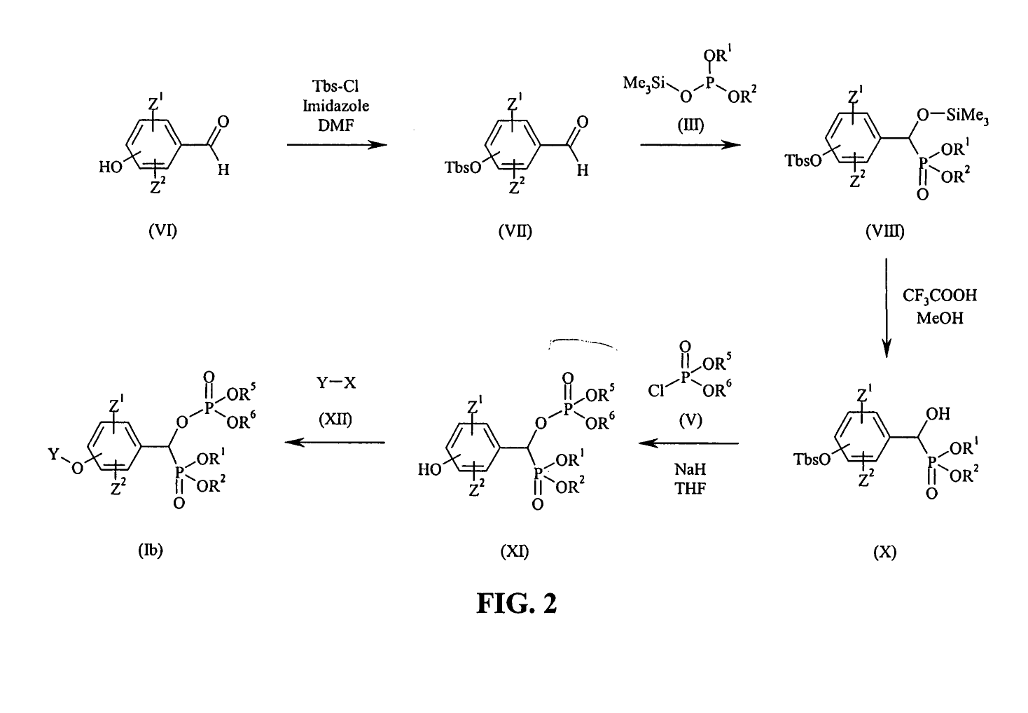

A second method, summarized in FIG. 2, comprises reacting the hydroxybenzaldehyde (VI) with t-butyldimethylsilyl chloride (Tbs-Cl) in presence of imidazole in dimethylformamide to give the Tbs protected hydroxybenzaldehyde (VII). Reaction with dialkyl trimethylsilyl phosphite (III) provides the corresponding α-trimethylsilyloxy phosphonate (VIII). Treating the latter with trifluoroacetic acid gives the α-hydroxyphosphonate (IX) which is reacted with dialkyl chlorophosphate (V) to give the Tbs protected phosphonophosphate (X). Deprotection of (X) yields the phenol-phosphonophosphate (XI). This latter serves as the intermediate in the preparation of the compounds (Ib) by means of a Mitsunobu or a Williamson reaction. In the Mitsunobu reaction the phenol (XI) is reacted with the primary alcohol (XII), wherein X═OH, in presence of a mixture of dialkyl azodicarboxylate and triphenylphosphine. In the Williamson the phenol (X) is reacted with the alkyl halide (XII), wherein X=halide, in presence of a base.

V. Determination of Biological Activity

The hydroxyphosphonates (Ia) and phosphonophosphates (Ib) of the invention can modulate (increase or decrease) the apoE production in plasma and in tissues. The activities of the compounds can be determined in an in vitro cell assay consisting in determining the effect of the test compound in modulating the secretion of apoE by an apoE secreting cell line (e.g., a monocyte-macrophage cell line such as the THP-1 cell line; a liver derived cell line such as the HepG2 cell line, an intestinal derived cell line such as the CaCo2 cell line or a brain derived cell line such as the astrocytoma CCF-STTG1 cell line).

### EXAMPLES OF THE INVENTION

Without further elaboration, it is believed that one skilled in the art can, using the preceding description, utilize the present invention to its fullest extent. The following specific examples are intended merely to illustrate the invention and not to limit the scope of the disclosure or the scope of the claims in any way whatsoever.

All new products were purified by flash chromatography employing silica gel 60 Fluka 60752; analytical TLC on silica gel 60 F₂₅₄ aluminium sheets from Merck. Detection by UV light at 254 nm.

The purity of all new products was determined by GC (BP6890 or HP5890, optima5, t_{R} in min.)

NMR spectra were performed using a Bruker AMX-400 (1H at 400 MHz) or Bruker AMX-500 (¹H at 500 MHz). Chemical shifts δ in ppm with respect to SiMe₄ (δ=0 ppm, internal reference). For ³¹P-NMR, chemical shifts δ in ppm with respect to H₃PO₄ (δ=0 ppm, external reference 85% H₃PO₄ in H₂O). Coupling constants in Hz. Nonobvious signal assignment was made by comparison with the spectra of the described similar compounds.

MS were performed using Varian CH4 or SM1 spectrometer with electron impact or electrospray, m/z (% of base peak).

IR spectra were performed using a Perkin Elmer Paragon 1000 FT-IR spectrometer. KBr for solids and neat for oil or liquids. Absorption bands in cm⁻¹.

With few exceptions, reagents and solvents were purchased from commercial suppliers and used without further purification. All reactions were conducted under N₂. Reaction temperatures refer to that of the heating bath. Reactions carried out with the exclusion of light were performed in flasks completely wrapped in aluminium foil.

All reactions were monitored by TLC and/or GC upon total consumption of the starting material.

### Example 1

### Diethyl 1-(diethoxy-phosphinyloxy)-1-(3-hydroxy-phenyl)-methylphosphonate

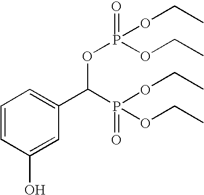

Imidazole (2.63 g, 185.52 mmol) was added portionwise under nitrogen at 0° C. to a mixture of 7.41 g (60.68 mmol) of 3-hydroxybenzaldehyde and 13.88 g (92.09 mmol) of tert-butyldimethylsilyl chloride in 60 ml DMF. The resulting mixture was stirred at room temperature for 2.5 h (GC monitoring), then the reaction mixture was poured into a mixture of ice (100 g) and hexane (100 ml). The aqueous solution was extracted with hexane (2×100 ml). The organics layers were combined together, washed with water (200 ml) and saturated NaCl solution (2×150 ml) and dried over MgSO₄. The residue (16.87 g) was purified by flash chromatography (Silica gel 60, AcOEt:Hexane 10:90, R_{f} 0.35) to give 12.98 g (54.91 mmol, yield 90.49%) of 3-tert-butyldimethylsilyloxybenzaldehyde. Diethyl trimethylsilyl phosphite (5.02 g, 22.68 mmol) was added to 4.80 g (20.31 mmol) of protected 3-hydroxybenzaldehyde. The mixture was heated at 110° C. for 2.5 h, then the excess of reagent and side products were removed under vacuum. The crude α-trimethylsiloxy phosphonate was used without further purification in the next step. Trifluoroacetic acid (3 drops) was added to a solution (≦20.31 mmol) of α-trimethylsiloxy phosphonate in 30 ml methanol. The mixture was stirred at room temperature over 15 h. The crude α-hydroxy phosphonate was concentrated by rotary evaporator and used in the next step without further purification.

A solution of α-hydroxy phosphonate (≦20.31 mmol) in 20 ml THF was added to a stirred suspension of 1.65 g (41.25 mmol) of NaH (60% dispersion in mineral oil) in 20 ml THF under nitrogen at 0° C. The resulting mixture was stirred at 0° C. for 30 min, then a solution of 3.5 ml (24.34 mmol) of diethyl chlorophosphate in 10 ml THF was added dropwise. The reaction mixture was allowed to warm to room temperature and stirred for 3 h. The excess of NaH was destroyed by careful treatment with ethyl acetate. A saturated NaHCO₃ solution was then added and the mixture taken up into ethyl acetate and washed with water, saturated NaCl solution and dried over MgSO₄. After solvent removal using a rotary evaporator, the residue (9.22 g) was purified by flash chromatography (Silica gel 60, AcOEt, R_{f} 0.25) to give 4.97 g (8.96 mmol, yield 44.12%) of tert-butyldimethylsilyl protected phosphonophosphate.

A mixture of 4.97 g (8.96 mmol) of protected phosphonophosphate, 11.61 g (36.80 mmol) of tetra butylammonium fluoride trihydrate and 6.50 g of acetic acid (108.24 mmol) in 30 ml of THF was stirred overnight at room temperature. 150 ml of water and 9.10 g (108.32 mmol) of sodium bicarbonate were added and the mixture taken up into ethyl acetate (3×50 ml) and washed with water (50 ml), saturated NaCl solution (2×50 ml) and dried over MgSO₄. After solvent removal using a rotary evaporator the oily residue (4.10 g) was purified by flash chromatography (Silica gel 60, AcOEt:MeOH 95:5, R_{f} 0.34) to give 3.54 g (8.93 mmol, C₁₅H₂₆O₈P₂, M_{w}=396.32, oil, yield 99.67%) of diethyl 1-(diethoxy-phosphinyloxy)-1-(3-hydroxy-phenyl)-methylphosphonate.

¹H-NMR (CDCl₃, 500 MHz) δ=8.33, s br, 1H; δ=7.20, t, 1H, J=7.9; δ=7.11, d, 1H, J=1.7; δ=6.99, d, 1H, J=7.5; δ=6.83, d, 1H, J=8.1; δ=5.50, d×d, 1H, J=13.5, J=10.3; δ=4.19-3.88, m, 4×2H; δ=1.32, 1.28, 1.22 and 1.15, t, 4×3H, J=7.1.

MS (70 eV) 397 (6), 396 (M⁺, 35), 260 (16), 259 (100), 203 (11), 186 (11), 123 (18), 122 (12), 121 (26), 109 (42), 107 (35), 99 (12), 95 (13), 81 (24), 77 (11), 65 (12).

### Example 2

### Diethyl 1-hydroxy-1-{3-[3-N-phthalimido-propoxy]-phenyl}-methylphosphonate

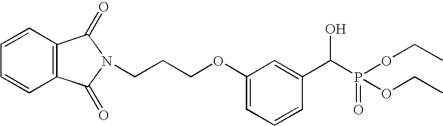

A solution of 12.12 g (43.85 mmol) of N-(3-bromopropyl)phthalimide in 50 ml 2-butanone was added dropwise to a stirred mixture of 5.59 g (43.49 mmol) of 3-hydroxybenzaldehyde, 8.49 g (61.43 mmol) of potassium carbonate and 1.32 g (3.97 mmol) of tetrabutyl ammonium bromide in 150 ml 2-butanone. The reaction mixture was warmed under reflux overnight then allowed to cool to room temperature. The final mixture was poured into water and taken up with AcOEt (3×50 ml). The organic layer was washed with saturated NaCl solution, dried over MgSO₄, filtered and then concentred by rotavapory evaporator. The residue (17.27 g) was purified by flash chromatography (Silica gel 60, AcOEt:Hexane 30:70, R_{f} 0.20) to give 7.29 g (23.57 mmol, yield 54.20%) of 3-(3-N-phthalimido-propoxy)-benzaldehyde.

Diethyl trimethylsilyl phosphite (1.62 g, 7.70 mmol) was added to 2.01 g (6.50 mmol) of 3-(3-N-phthalimido-propoxy)-benzaldehyde. The mixture was heated at 110° C. for 3 h, then the excess of reagent and side products were removed under vacuum. The crude α-trimethylsiloxyphosphonate was used without further purification in the next step.

Trifluoroacetic acid (6 drops) was added to a solution (≦6.50 mmol) of α-trimethylsiloxy phosphonate in 50 ml of methanol. The mixture was stirred overnight at room temperature. The crude α-hydroxy phosphonate was concentred by rotary evaporator. The oily residue (4.10 g) was purified by flash chromatography (Silica gel 60, AcOEt, R_{f} 0.24) to give 2.85 g (6.37 mmol, C₂₂H₂₆NO₇P, M_{w}=447.43, white solid, yield 98.00%) of diethyl 1-hydroxy-1-[3-(3-N-phthalimido-propoxy)-phenyl]-methylphosphonate.

¹H-NMR (CDCl₃, 500 MHz) δ=7.85-7.83, m, 2H; δ=7.73-7.71, m, 2H; δ=7.23, t, 1H, J=7.9; δ=7.04, d, 1H, J=7.6; δ=6.99, s br, 1H; δ=6.76, d, 1H, J=8.2; δ=4.96, d, 1H, J=11.0; δ=4.22-3.95, m, 3×2H; δ=3.91, t, 2H, J=7.0; δ=2.18, quint, 2H, J=6.7; δ=1.28 and 1.23, t, 2×3H, J=7.0.

MS (70 eV) 447 (M⁺, <1), 189 (13), 188 (100), 161 (12), 160 (61), 130 (20), 111 (18), 83 (19), 77 (15), 65 (16).

### Example 3

### Diethyl 1-(diethoxy-phosphinyloxy)-1-[3-(3-N-phthalimido-propoxy)-phenyl]-methylphosphonate

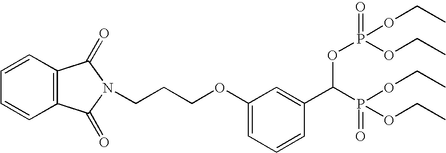

A solution of 2.52 g (9.40 mmol) of N-(3-bromopropyl)phthalimide in 20 ml 2-butanone was added dropwise to a stirred mixture of 3.30 g (8.33 mmol) of diethyl [(diethoxy-phosphinyloxy)-(3-hydroxy-phenyl)-methyl]-phosphonate (SR-123117), 1.78 g (12.88 mmol) of potassium carbonate and 0.35 g (1.05 mmol) of tetrabutyl ammonium bromide in 30 ml of the same solvent. The reaction mixture was warmed under reflux over 4.5 h then allowed to cool to room temperature. The final mixture was poured into water and taken up with CH₂Cl₂ (3×50 ml). The organic layer was washed with saturated NaCl solution, dried over MgSO₄, filtered and then concentred by rotavapory evaporator. The residue (5.37 g) was purified by flash chromatography (Silica gel 60, AcOEt:MeOH 98:2, R_{f} 0.20) to give 4.59 g (7.87 mmol, C₂₆H₃₅NO₁₀P₂, M_{w}=583.52, oil, yield 94.48%) of diethyl 1-(diethoxy-phosphinyloxy)-1-[3-(3-N-phthalimido-propoxy)-phenyl]-methylphosphonate.

¹H-NMR (CDCl₃, 500 MHz) δ=7.85-7.83, m, 2H; 5=7.74-7.72, m, 2H; δ=7.24, t, 1H, J=8.0; δ=7.07, d, 1H, J=7.2; δ=6.99, d, 1H, J=1.8; δ=6.78, d, 1H, J=8.0; δ=5.49, d×d, 1H, J=13.6, J=10.6; δ=4.20-3.85, m, 6×2H; δ=2.18, quint, 2H, J=6.5; δ=1.30-1.24, m, 2×3H; δ=1.23, t, 3H, J=7.1; δ=1.15, t×d, 3H, J=7.1, J=1.0.

¹³C-NMR (CDCl₃, 125 MHz) δ=168.3, (C); δ=158.6, (C); δ=135.0, (C); δ=133.9, (CH); δ=132.1, (C); δ=129.3, (CH); δ=123.2, (CH); δ=120.4, (CH), d, J=6; δ=115.4, (CH), d, J=2; δ=113.7, (CH), d, J=6; δ=74.6, (CH), d×d, J=171, J=7; δ=65.6, (CH₂); δ=64.1-63.4, (CH₂), m; δ=35.4, (CH₂); δ=28.2, (CH₂); δ=16.4-15.8, (CH₃), m.

MS (70 eV) 584 (15), 583 (M⁺, 48), 447 (21), 446 (70), 429 (10), 189 (35), 188 (100), 161 (16), 160 (72), 130 (32), 121 (11), 109 (35), 81 (24), 77 (12), 65 (11).

### Example 4

### Diethyl 1-(diethoxy-phosphinyloxy)-1-(3-{3-[3-(2-hydroxy-phenyl)-propionylamino]-propoxy}-phenyl)-methylphosphonate

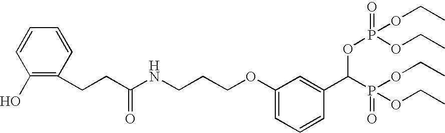

A mixture of 2.50 g (4.28 mmol) of diethyl 1-(diethoxy-phosphinyloxy)-1-[3-(3-N-phthalimido-propoxy)-phenyl]-methylphosphonate (SR-137517) and 1.80 g (8.63 mmol) of hydrazine hydrate (−25% in water) was refluxed over 3 h. The solvent was removed and the residue was taken-up with AcOEt. The insoluble phthalylhydrazide was removed by filtration over celite. The crude diethyl I-(diethoxyphosphinyloxy)-1-[3-(3-amino-propoxy)-phenyl]-methylphosphonate was concentred by rotary evaporator (1.60 g, 3.53 mmol, yield 82.48%) and used in the next step without further purification.

To a stirred solution of 1.60 g (3.53 mmol) of diethyl 1-(diethoxy-phosphinyloxy)-1-[3-(3-amino-propoxy)-phenyl]-methylphosphonate in 10 ml EtOH was added dropwise a solution of 0.60 g (4.01 mmol) of dihydrocoumarin in 5 ml EtOH. The mixture was stirred overnight at room temperature. The solvent was removed and the residue was purified by flash chromatography (Silica gel 60, AcOEt:MeOH 95:5, R_{f} 0.27) to give 0.96 g (1.60 mmol, C₂₇H₄₁NO₁₀P₂, M_{w}=601.58, oil, yield 45.33%) of diethyl 1-(diethoxy-phosphinyloxy)-1-(3-{3-[3-(2-hydroxy-phenyl)-propionylamino]-propoxy}-phenyl)-methylphosphonate.

¹H-NMR (CDCl₃, 500 MHz) δ=9.0-8.8, s br, 1H; δ=7.29, t, 1H, J=7.9; δ=7.09-7.06, m, 3×1H; δ=7.00, t×d, 1H, J=7.8, J=1.6; δ=6.84, d, 1H, J=8.2; δ=6.79, d, 1H, J=7.9; δ=6.76, t×d, 1H, J=7.5, J=1.3; δ=6.5-6.4, s br, 1H; δ=5.56, d×d, 1H, J=13.9, J=10.4; δ=4.20-3.82, m, 5×2H; δ=3.39-3.36, m, 2H; δ=2.94, t, 2H, J=6.9; δ=2.60, t, 2H, J=7.0; δ=1.85-1.78, m, 2H; δ=1.32, t, 3H, J=7.2; δ=1.30, t×d, 3H, J=7.3, J=1.0; δ=1.24, t, 3H, J=7.1; δ=1.16, t×d, 3H, J=7.3, J=1.0.

MS (70 eV) 601 (M⁺, 8), 396 (8), 327 (6), 310 (7), 282 (7), 259 (9), 244 (18), 188 (6), 162 (10), 155 (8), 149 (15), 148 (91), 147 (10), 127 (12), 121 (18), 120 (90), 119 (18), 112 (11), 109 (19), 107 (18), 106 (14), 99 (24), 92 (17), 91 (60), 86 (100), 84 (19), 82 (10), 81 (25), 78 (47), 77 (17), 72 (23), 65 (15), 63 (13), 58 (40), 57 (10), 56 (20), 52 (14), 51 (41), 50 (14).

IR 3296 (s br), 2984 (m), 2935 (m), 1736 (m), 1648 (s), 1601 (w), 1560 (w), 1490 (w), 1457 (m), 1732 (w), 1263 (s), 1163 (w), 1099 (w), 1029 (s), 980 (m), 877 (w), 800 (w), 755 (m), 695 (w), 610 (w), 501 (m).

### Example 5

### Dimethyl 1-(diethoxy-phosphinyloxy)-1-[4-(pyridin-2-yl-methoxy)-phenyl]-methylphosphonate

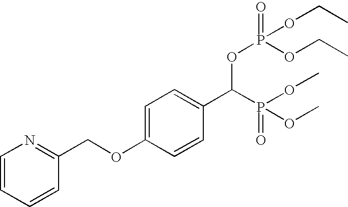

A solution of 5.52 g (50.58 mmol) of pyridin-2-yl-methanol in 50 ml DMF was added under nitrogen at 0° C. to a stirred suspension of 2.76 g (69.0 mmol) of NaH (60% dispersion in mineral oil) in 50 ml DMF. The mixture was stirred at 0° C. until the vigourous reaction ceased, then a solution of 6.0 ml (56.85 mmol) of 4-fluorobenzaldehyde in 50 ml DMF was added dropwise. The reaction mixture was stirred at room temperature over 5 h. The excess of NaH was destroyed by careful treatment with ethyl acetate. A mixture ice-water was then added and the mixture extracted with AcOEt (4×300 ml) and washed with water, saturated NaCl solution and dried over MgSO₄. After solvent removal, the residue (15.43 g) was purified by flash chromatography (Silica gel 60, AcOEt:Hexane 80:20 R_{f} 0.22) to give 6.44 g (32.08 mmol, yield 63.42%) of 4-(pyridin-2-ylmethoxy)-benzaldehyde.

Dimethyl trimethylsilyl phosphite (2.14 g, 11.16 mmol) was added to 2.28 g (10.69 mmol) of 4-(pyridin-2-ylmethoxy)-benzaldehyde. The mixture was heated at 110° C. over 2 h, then the excess of reagent and side products were removed under vacuum. The crude dimethyl {1-trimethylsiloxy-[4-(pyridin-2-ylmethoxy)-phenyl]-methyl}-phosphonate was used without further purification in the next step.

Trifluoroacetic acid (3 drops) was added to a solution (≦10.69 mmol) of α-trimethylsiloxy phosphonate in 30 ml methanol. The mixture was stirred at room temperature overnight. The crude dimethyl {1-hydroxy-[4-(pyridin-2-ylmethoxy)-phenyl]-methyl}-phosphonate was concentred by rotary evaporator and used in the next step without further purification.

A solution of α-hydroxy phosphonate (≦10.69 mmol) in 20 ml THF was added to a stirred suspension of 0.86 g (21.50 mmol) of NaH (60% dispersion in mineral oil) in 20 ml THF under nitrogen at 0° C. The resulting mixture was stirred at 0° C. for 30 min, then a solution of 1.8 ml (12.02 mmol) of diethyl chlorophosphate in 10 ml THF was added dropwise. The reaction mixture was allowed to warm to room temperature and stirred overnight.

The excess of NaH was destroyed by careful treatment with ethyl acetate (3×100 ml). A saturated NaHCO₃ solution was then added and the mixture taken up into ethyl acetate and washed with water, saturated NaCl solution and dried over MgSO₄. After solvent removal using a rotary evaporator, the residue (5.57 g) was purified by flash chromatography (Silica gel 60, AcOEt:MeOH 90:10, R_{f} 0.24) to give 1.42 g (3.09 mmol, C₁₉H₂₇NO₈P₂, M_{w}=459.38, oil, yield 28.91%) of dimethyl 1-(diethoxy-phosphinyloxy)-1-[4-(pyridin-2-ylmethoxy)-phenyl]-methylphosphonate.

¹H-NMR (CDCl₃, 500 MHz) δ=8.61, d, 1H, J=4.7; δ=7.72, t×d, 1H, J=7.7, J=1.7; δ=7.51, d, 1H, J=7.8; δ=7.47, d×d, 2H, J=8.7, J=1.8; δ=7.24, d×d, 1H, J=7.4, J=5.1; δ=7.01, d, 2H, J=8.7; δ=5.54, d×d, 1H, J=13.0, J=10.5; δ=5.21, s, 2H; δ=4.18-3.82, m, 2×2H; δ=3.78 and 3.67, d, 2×3H, J=10.7; δ=1.29 and 1.12, t, 2×3H, J=7.1.

MS (70 eV) 460 (2), 459 (M⁺, 8), 367 (12), 351 (20), 350 (100), 306 (12), 214 (34), 198 (16), 149 (11), 141 (34), 127 (14), 121 (10), 113 (50), 109 (28), 99 (21), 96 (14), 95 (29), 93 (77), 92 (98), 82 (12), 81 (25), 79 (10), 65 (30), 45 (12).

### Example 6

### Exemplary Hydroxyphosphonate Compounds of Formula (Ia) as Prepared by the Method as Shown in FIG. 1

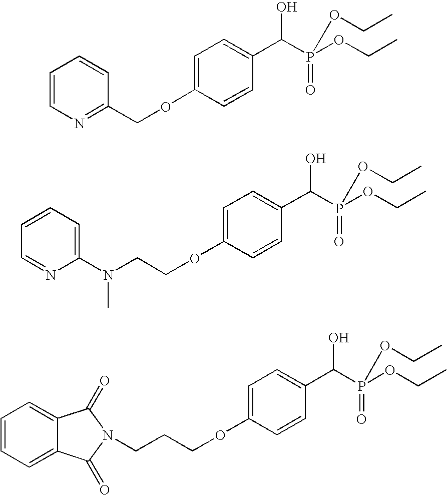

### Example 7

### Exemplary Phosphonophosphates of Formula (Ib) Prepared by the Method as Shown in FIG. 1

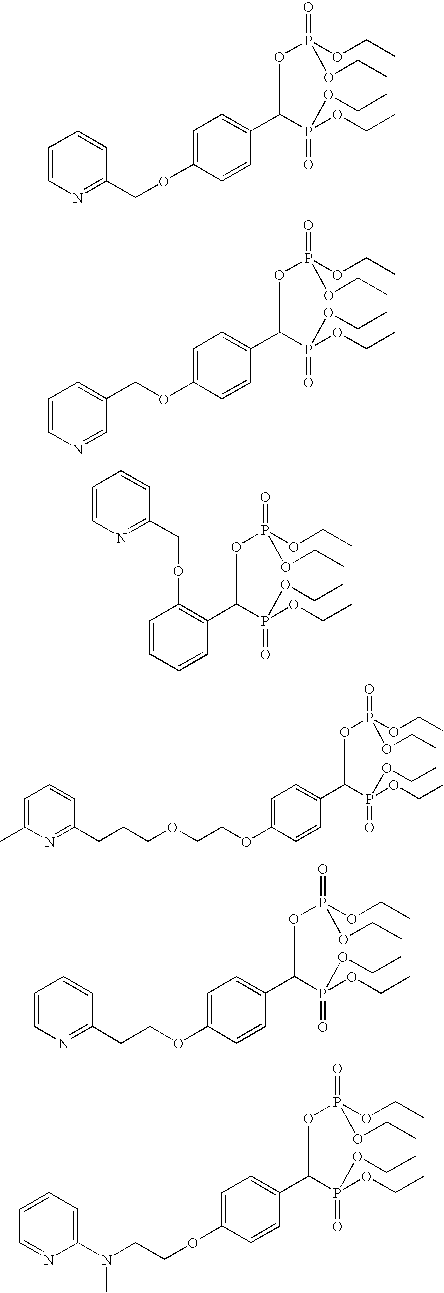

### Example 8

### Exemplary Phosphonophosphates of Formula (Ib) Prepared by the Method as Shown in FIG. 2

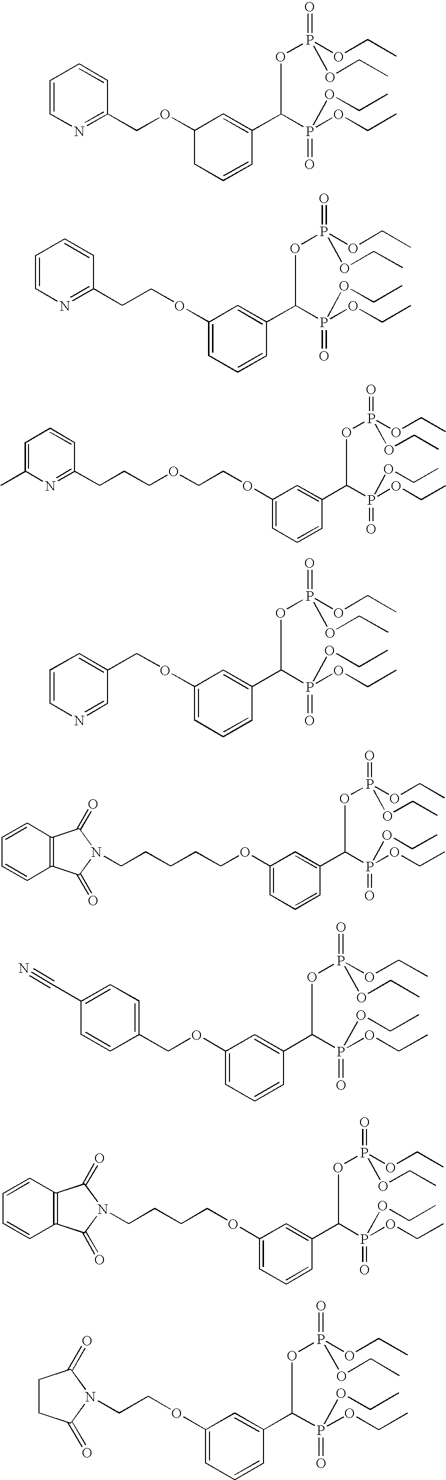

### Example 9

### Biological Activity

A. Methods

THP-1 cell line (ATCC TIB-202) are cultured in RPMI 1640 (with 2 mM of L-glutamine and 2 g/l of glucose, Invitrogen) supplemented with 2 g/l of sodium hydrogen carbonate (Fluka), 10 U/ml of penicillin, 10 μg/ml of streptomycin (Penicillin-Streptomycin, Invitrogen), 20 μM of 2-mercaptoethanol (Fluka) and 10% FCS (Amimed). Cells are incubated at 37° C. and 5% CO₂.

Cells are seeded in 24 well tissue culture plates (Falcon), 2×10⁵ cells in 500 μl of culture medium per well or 6 well plates for RT-PCR purpose, 1×10⁶ cells in 2.5 ml of medium. PMA (Phorbol 12-myristate 13-acetate, Alexis), (stock in DMSO (Fluka) at 1 mg/ml) and other compounds are diluted in ethanol and added to the cells at an ethanol final concentration not exceeding 1%. Same volume of ethanol is added in controls. Plates are incubated for 3 days at 37° C. and 5% CO₂ to allow cell differentiation and apoE secretion.

Medium contained in the wells is harvested and centrifuged 5 min at 1200 rpm. Supernatants are store at −20° C. until apoE quantification. The cell pellets are resuspended in PBS and counted with a Z1 Coulter Counter. After a wash with PBS, adherent cells in the well bottoms are detached with trypsin-EDTA solution 10× (Invitrogen) diluted in PBS. The reaction is stopped with the addition of culture medium and the detached cells are counted.

ApoE was quantified by ELISA. 96 well plates (Costar) are coated with 5% of gelatine (from porcine skin, Fluka) in carbonate-bicarbonate buffer (Sigma) for 1 hour at 37° C. After removing the coating solution, 100 μl of THP-1 supernatant are added per well, diluted 5 times in buffer (PBS, 1% Top-Block (Juro), 0.1% Tween 20 (Fluka)). The incubation last 1 hour at 37° C., then the wells are washed 3 times with 200 μl of buffer. Plates are incubated for 1 hour at 37° C. with continuous stirring with the primary antibody (goat anti-human apoE IgG, Calbiochem) at a 10000-time dilution in buffer, 100 μl per well. After 3 washes, plates are incubated with the secondary antibody (rabbit anti-goat-IgG peroxidase conjugated, Sigma) diluted 5000 times, 100 μl per well, at 37° C. with shaking. Then the wells are washed 5 more times and the detection is achieved by adding 100 μl per well of o-phenylenediamine dihydrochloride (Sigma) and incubated for 15-20 minutes with shaking at room temperature. When the appropriated colour is reached, the reaction is stopped by adding 50 μl per well of 3 M sulfuric acid (Fluka) with shaking for 1 minute at room temperature. The absorbance is read at 492 nm versus 620 nm with a microplate photometer (Anthos Reader 2001).

B. Results

TABLE 1
Phosphonophosphates

| | | % ApoE Change | |
|---|---|---|---|
| | | 1.25 nM | 2.5nM |
| Compound | μM | PMA | PMA |
| Diethyl 1-(diethoxy-phosphinyloxy)-1-[4-(2-pyridin-2- | 25 | 177 | 154 |
| yl-ethoxy)-phenyl]-methylphosphonate | 50 | 432 | 290 |
| | 100 | 1239 | 400 |
| Diethyl 1-(diethoxy-phosphinyloxy)-1-[4-(pyridin-2-yl- | 25 | 112 | 98 |
| methoxy)-phenyl]-methylphosphonate | 50 | 452 | 135 |
| | 100 | 779 | 266 |
| Diethyl 1-(diethoxy-phosphinyloxy)-1-{4-[2-(3-{6- | 25 | — | 495 |
| methyl-pyridin-2-yl}-propoxy)-ethoxy]-phenyl}- | 50 | — | 653 |
| methylphosphonate | 100 | — | 746 |
| Diethyl 1-(diethoxy-phosphinyloxy)-1-[4-(pyridin-3-yl- | 25 | 95 | 179 |
| methoxy)-pheny]-methylphosphonate | 50 | 189 | 245 |
| | 100 | 535 | 434 |
| Dimethyl 1-(diethoxy-phosphinyloxy)-1-[4-(pyridin-2- | 25 | 53 | 107 |
| yl-methoxy)-phenyl]-methylphosphonate | 50 | 96 | 153 |
| | 100 | 263 | 250 |
| Diethyl 1-(diethoxy-phosphinyloxy)-1-(biphenyl-4-yl)- | 25 | 172 | 138 |
| methyiphosphonate | 50 | 198 | 119 |
| | 100 | 142 | 71 |
| Diethyl 1-(diethoxy-phosphinyloxy)-1-{4-[2-(methyl- | 25 | 205 | 184 |
| pyridin-2-yl-amino)-ethoxy]-phenyl)- | 50 | 160 | 125 |
| methylphosphonate | 100 | 52 | −9 |
| Diethyl 1-(diethoxy-phosphinyloxy)-1-(4-hexyloxy- | 25 | 64 | 81 |
| phenyl)-methylphosphonate | 50 | 173 | 133 |
| | 100 | 101 | −4 |
| Diethyl 1-(diethoxy-phosphinyloxy)-1-(4-benzyloxy- | 25 | −6 | 35 |
| phenyl)-methylphosphonate | 50 | 118 | 81 |
| | 100 | 171 | 120 |
| Diethyl 1-(diethoxy-phosphinyloxy)-1-[4-(diethoxy- | 25 | 52 | 59 |
| phosphinyloxy)-phenyl]-methylphosphonate | 50 | 99 | 62 |
| | 100 | 171 | 127 |
| Diethyl 1-(diethoxy-phosphinyloxy)-1-[4-(3-methoxy- | 25 | −19 | 7 |
| 4,5-methylenedioxy)-phenyl]-methylphosphonate | 50 | 3 | −5 |
| | 100 | 33 | 40 |
| Diethyl 1-(diethoxy-phosphinyloxy)-1-{3-[2-(3-{6- | 25 | — | 340 |
| methyl-pyridin-2-yl}-propoxy)-ethoxy]-phenyl}- | 50 | — | 491 |
| methylphosphonate | 100 | — | 811 |
| Diethyl 1-(diethoxy-phosphinyloxy)-1-[3-(3-N- | 25 | 581 | 192 |
| phthalimido-propoxy)-phenyl]-methylphosphonate | 50 | 741 | 200 |
| | 100 | 793 | 195 |
| Diethyl 1-(diethoxy-phosphinyloxy)-1-[3-(5-N- | 25 | 3375 | 1196 |
| phthalimido-pentoxy)-phenyl]-methylphosphonate | 50 | 1526 | 310 |
| | 100 | −93 | −92 |
| Diethyl 1-(diethoxy-phosphinyloxy)-1-[3-(2-pyridin-2- | 25 | 123 | 124 |
| yl-ethoxy)-phenyl]-methylphosphonate | 50 | 259 | 216 |
| | 100 | 807 | 385 |
| Dietbyl 1-(diethoxy-phosphinyloxy)-1-[3-(pyridin-3-yl- | 25 | 52 | 15 |
| methoxy)-phenyl]-methylphosphonate | 50 | 551 | 6 |
| | 100 | 1351 | 98 |
| Diethyl 1-(diethoxy-phosphinyloxy)-1-[3-(pyridin-2-yl- | 25 | 59 | 29 |
| methoxy)-phenyl]-methylphosphonate | 50 | 101 | 66 |
| | 100 | 130 | 243 |
| Diethyl 1-(diethoxy-phosphinyloxy)-1-[3-phenoxy- | 25 | 192 | 103 |
| phenyl]-methylphosphonate | 50 | 269 | 63 |
| | 100 | 105 | 22 |
| Diethyl 1-(diethoxy-phosphinyloxy)-1-[3-(4-cyano- | 25 | 80 | 43 |
| benzyloxy)-phenyl]-methylphosphonate | 50 | −72 | −84 |
| | 100 | −88 | −94 |

* = 5.0 nM PMA.

TABLE 2
Hydroxphosphonates

| | | % ApoE Change | |
|---|---|---|---|
| | | 1.25 nM | 2.5nM |
| Compound | μM | PMA | PMA |
| Diethyl 1-hydroxy-1-{4-[3-N-phthalimido-propoxy]- | 25 | 543 | 304 |
| phenyl}-methylphosphonate | 50 | 961 | 396 |
| | 100 | 1436 | 465 |
| Diethyl 1-hydroxy-1-{4-[2-(methyl-pyridin-2-yl- | 25 | 294 | 132 |
| amino)-ethoxy]-phenyl}-methylphosphonate | 50 | 384 | 154 |
| | 100 | 1069 | 353 |
| Diethyl 1-hydroxy-1-{3-[3-N-phthalimido-propoxy]- | 25 | 59 | 42 |
| phenyl}-methylphosphonate | 50 | 140 | 74 |
| | 100 | 217 | 94 |
| Diethyl 1-hydroxy-1-[4-(pyridin-2-yl-methoxy)-phenyl]- | 25 | −9 | 0 |
| methlyphosphonate | 50 | 24 | 3 |
| | 100 | 76 | 100 |

### REFERENCES

The following references, to the extent that they provide exemplary procedural or other details supplementary to those set forth herein, are specifically incorporated herein by reference.
Berge et al., “Pharmaceutical salts,” J. Pharm. Sci., 66:1-19, 1997
Gaillard, “Apolipoprotein E intrathecal synthesis is decreased in multiple sclerosis patients,” Ann. Clin. Biochem., 33:148-150, 1996.
Laskowitz et al., “Apolipoprotein E-deficient mice have increased susceptibility to focal cerebral ischemia,” J. Cerebral Blood Flow Metab., 17: 753-758 1997.
Leininger-Muller & Siest, “The rat, a useful model for pharmacological studies on apolipoprotein E,” Life Sciences, 58:455-467, 1996.
Linton et al., “Prevention of atherosclerosis in apolipoprotein E-deficient mice by bone marrow transplantation” Science, 267:1034-1037, 1995.
Oitzl et al., “Severe learning deficits in apolipoprotein E knockout mice in a water maze task,”Brain Res., 752:189-196, 1997.
Poirier, “Apolipoprotein E in animal models of CNS injury and in Alzheimer's disease,” Trends in Neurosciences 17:525-530, 1994.
Plump, “Atherosclerosis and the mouse—a decade of experience,” Annal. Med., 29:193-198, 1997.
Richard et al., “Identification of a new apolipoprotein E variant (E(2) Arg(142)→Leu) in type III hyperlipidemia” Atherosclerosis, 112:19-28, 1995.
Roselaar & Daugherty, “Apolipoprotein E-deficient mice have impaired innate immune responses to listeria monocytogenes in vivo,” J. Lipid Res., 39:1740-1743, 1998.
Rubinsztein, “Apolipoprotein E—a review of its roles in lipoprotein metabolism, neuronal growth and repair and as a risk factor for Alzheimer's disease,” Psychological Med., 25:223-229, 1995.
Srivastava et al., “Estrogen up-regulates apolipoprotein E (ApoE) gene expression by increasing apoE mRNA in the translating pool via the estrogen receptor alpha-mediated pathway” J. Biol. Chem., 272:33360-33366, 1997.
Stone et al., “Astrocytes and microglia respond to estrogen with increased apoE mRNA in vivo and in vitro” Experimental Neurology, 143:313-318, 1997.
van Vlijmen et al., “Apolipoprotein E*3 Leiden transgenic mice as a test model for hypolipidaemic drugs,” Arzneimittel-Forschung, 48:396-402, 1998.
Walker et al., “Cerebral lipid deposition in aged Apolipoprotein E-deficient mice”, Am. J. Pathol., 151:1371-1377, 1997.

1. A phosphonate derivative of the formula: 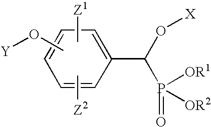
wherein:
Y is hydrogen, aryl, C₂-C₆ alkyl, PO(OR⁵OR⁶), R³R⁴N(CH₂)— or A-L;
A is aryl or heterocycle;
L is —(CH₂)ᵣ—, —(CH₂)ₚO(CH₂)_{q}—, —(CH₂)ₚN(R⁷)(CH₂)_{q}— or —(CH₂)ₚNHCO(CH₂)_{q}— wherein R⁷ is hydrogen, C₁-C₄ alkyl, aryl or C₁-C₃ cyanoalkyl;
m, p, q and r independently are an integer from 0 to 6;
X is hydrogen or PO(OR⁵OR⁶);
R¹, R² and R⁵, R⁶ are independently hydrogen or C₁-C₆ alkyl;
R³ and R⁴ are independently hydrogen, C₁-C₄ allyl;
and Z¹ and Z² are independently hydrogen, C₁-C₄ alkyl, or C₁-C₄ alkoxy;
with the proviso that when X is hydrogen, then Y—O—, Z¹ and Z² are not all independently hydroxy, hydrogen, alkoxy or alkyl;
or a pharmaceutically acceptable salt thereof.

2. The phosphonate derivative of claim 1, wherein and Z¹ and Z² are hydrogen.

3. The phosphonate derivative, wherein Y is hydrogen, aryl, or diethoxyphosphinyl.

4. The phosphonate derivative, wherein A is pyridin-2-yl, 5-methyl-pyridin-2-yl, pyridin-3-yl, N-phthalimido, phenyl-4-yl or p-cyanophenyl.

5. The phosphonate derivative of claim 1, wherein A is pyridinyl-2-yl.

6. The phosphonate derivative of claim 1, wherein A is pyridinyl-3-yl.

7. The phosphonate derivative of claim 1, wherein A is N-phthalimido.

8. The phosphonate derivative of claim 1, wherein R¹, R² and R⁵, R⁶ are independently methyl or ethyl.

9. The phosphonate derivative of claim 1, wherein said phosphonate derivative is diethyl 1-hydroxy-1-{4-[3-N-phthalimido-propoxy]-phenyl}-methylphosphonate.

10. The phosphonate derivative of claim 1, wherein said phosphonate derivative is diethyl 1-(diethoxy-phosphinyloxy)-1-[3-(pyridin-3-yl-methoxy)-phenyl]-methylphosphonate.

11. The phosphonate derivative of claim 1, wherein said phosphonate derivative is diethyl 1-(diethoxy-phosphinyloxy)-1-[4-(2-pyridin-2-yl-ethoxy)-phenyl]-methylphosphonate.

12. The phosphonate derivative of claim 1, wherein said phosphonate derivative is diethyl 1-hydroxy-1-{4-[2-(methyl-pyridin-2-yl-amino)-ethoxy]-phenyl}-methylphosphonate.

13. The phosphonate derivative of claim 1, wherein said phosphonate derivative is diethyl 1-(diethoxy-phosphinyloxy)-1-[3-(3-N-phthalimido-propoxy)-phenyl]-methylphosphonate.

14. The phosphonate derivative of claim 1, wherein said phosphonate derivative is diethyl 1-(diethoxy-phosphinyloxy)-1-[3-(5-N-phthalimido-pentoxy)-phenyl]-methylphosphonate.

15. The phosphonate derivative of claim 1, wherein said phosphonate derivative is diethyl 1-(diethoxy-phosphinyloxy)-1-[3-(2-pyridin-2-yl-ethoxy)-phenyl]-methylphosphonate.

16. A pharmaceutical composition comprising a phosphonate derivative according to claim 1 and a carrier.

17. The pharmaceutical composition of claim 16, wherein A is pyridinyl-2-yl.

18. The pharmaceutical composition of claim 16, wherein A is pyridinyl-3-yl.

19. The pharmaceutical composition of claim 16, wherein A is N-phthalimido.

20. A method of modulating the production of apoE by an apoE producing cell, comprising contacting said apoE producing cell with an effective amount of a phosphonate derivative of the formula: 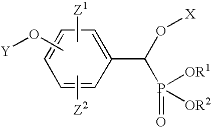
wherein:
Y is hydrogen, aryl, C₂-C₆ alkyl, PO(OR⁵OR⁶), R³R⁴N(CH₂)ₘ— or A-L;
A is aryl or heterocycle;
L is —(CH₂)ᵣ—, —(CH₂)ₚO(CH₂)_{q}—, —(CH₂)ₚN(R⁷)(CH₂)_{q}— or —(CH₂)ₚNHCO(CH₂)_{q}— wherein R⁷ is hydrogen, C₁-C₄ alkyl, aryl or C₁-C₃ cyanoalkyl;
m, p, q and r independently are an integer from 0 to 6;
X is hydrogen or PO(OR⁵OR⁶);
R¹, R² and R⁵, R⁶ are independently hydrogen or C₁-C₆ alkyl;
R³ and R⁴ are independently hydrogen, C₁-C₄ alkyl;
and Z¹ and Z² are independently hydrogen, C₁-C₄ alkyl, or C₁-C₄ alkoxy;
with the proviso that when X is hydrogen, then Y—O—, Z¹ and Z² are not all independently hydroxy, hydrogen, alkoxy or alkyl;
or a pharmaceutically acceptable salt thereof.

21. The method of claim 20, wherein and Z¹ and Z² are hydrogen.

22. The method of claim 20, wherein Y is hydrogen, aryl, or diethoxyphosphinyl.

23. The method of claim 20, wherein A is pyridin-2-yl, 5-methyl-pyridin-2-yl, pyridin-3-yl, N-phthalimido, phenyl-4-yl or p-cyanophenyl.

24. The method of claim 20, wherein R¹, R² and R⁵, R⁶ are independently methyl or ethyl.

25. The method of claim 20, wherein said phosphonate derivative is diethyl 1-hydroxy-1-{4-[3-N-phthalimido-propoxy]-phenyl}-methylphosphonate, diethyl 1-(diethoxy-phosphinyloxy)-1-[3-(pyridin-3-yl-methoxy)-phenyl]-methylphosphonate, diethyl 1-(diethoxy-phosphinyloxy)-1-[3-(2-pyridin-2-yl-ethoxy)-phenyl]-methylphosphonate, diethyl 1-(diethoxy-phosphinyloxy)-1-[4-(2-pyridin-2-yl-ethoxy)-phenyl]-methylphosphonate, diethyl 1-hydroxy-1-{4-[2-(methyl-pyridin-2-yl-amino)-ethoxy]-phenyl}-methylphosphonate, diethyl 1-(diethoxy-phosphinyloxy)-1-[3-(3-N-phthalimido-propoxy)-phenyl]-methylphosphonate, or diethyl 1-(diethoxy-phosphinyloxy)-1-[3-(5-N-phthalimido-pentoxy)-phenyl]-methylphosphonate.

26. The method of claim 20, wherein said modulating the production of apoE increase the production of apoE.

27. The method of claim 20, wherein said modulating the production of apoE decreases the production of apoE.

28. A method of modulating apoE levels in a patient in need of such treatment, comprising administration of an effective amount of a phosphonate derivative of the formula: 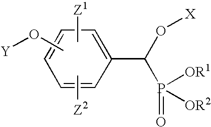
wherein:
Y is hydrogen, aryl, C₂-C₆ alkyl, PO(OR⁵OR⁶), R³R⁴N(CH₂)ₘ— or A-L;
A is aryl or heterocycle;
L is —(CH₂)ᵣ—, —(CH₂)ₚO(CH₂)_{q}—, —(CH₂)ₚN(R⁷)(CH₂)_{q}— or —(CH₂)ₚNHCO(CH₂)_{q}— wherein R⁷ is hydrogen, C₁-C₄ alkyl, aryl or C₁-C₃ cyanoalkyl;
m, p, q and r independently are an integer from 0 to 6;
X is hydrogen or PO(OR⁵OR⁶);
R¹, R² and R⁵, R⁶ are independently hydrogen or C₁-C₆ alkyl;
R³ and R⁴ are independently hydrogen, C₁-C₄ alkyl;
and Z¹ and Z² are independently hydrogen, C₁-C₄ alkyl, or C₁-C₄ alkoxy;
with the proviso that when X is hydrogen, then Y—O—, Z¹ and Z² are not all independently hydroxy, hydrogen, alkoxy or alkyl;
or a pharmaceutically acceptable salt thereof.

29. The method of claim 28, wherein and Z¹ and Z² are hydrogen.

30. The method of claim 28, wherein Y is hydrogen, aryl, or diethoxyphosphinyl.

31. The method of claim 28, wherein A is pyridin-2-yl, 5-methyl-pyridin-2-yl, pyridin-3-yl, N-phthalimido, phenyl-4-yl or p-cyanophenyl.

32. The method of claim 28, wherein R¹, R² and R⁵, R⁶ are independently methyl or ethyl.

33. The method of claim 28, wherein said phosphonate derivative is diethyl 1-hydroxy-1-{4-[3-N-phthalimido-propoxy]-phenyl}-methylphosphonate, diethyl 1-(diethoxy-phosphinyloxy)-1-[3-(pyridin-3-yl-methoxy)-phenyl]-methylphosphonate, diethyl 1-(diethoxy-phosphinyloxy)-1-[3-(2-pyridin-2-yl-ethoxy)-phenyl]-methylphosphonate, diethyl 1-(diethoxy-phosphinyloxy)-1-[4-(2-pyridin-2-yl-ethoxy)-phenyl]-methylphosphonate, diethyl 1-hydroxy-1-{4-[2-(methyl-pyridin-2-yl-amino)-ethoxy]-phenyl}-methylphosphonate, diethyl 1-(diethoxy-phosphinyloxy)-1-[3-(3-N-phthalimido-propoxy)-phenyl]-methylphosphonate, or diethyl 1-(diethoxy-phosphinyloxy)-1-[3-(5-N-phthalimido-pentoxy)-phenyl]-methylphosphonate.

34. The method of claim 28, wherein said modulation of said apoE levels in said patient comprises increasing said apoE levels.

35. The method of claim 34, wherein said patient is suffering from atherosclerosis, Alzheimer's disease, macular degeneration, retinitis pigmentosa, stroke, degenerative neuropathy, xanthoma or xanthelasma.

36. The method of claim 35, wherein said degenerative neuropathy is associated with diabetic neuropathy or multiple sclerosis.

37. The method of claim 28, wherein said modulation of said apoE levels in said patient comprises decreasing said apoE levels.

38. The method of claim 37, wherein said patient expresses apoE4, apoE Leiden or a non-functional mutant form of apoE.

39. The method of claim 38, wherein said patient is suffering from atherosclerosis or Alzheimer's disease.

40. A method of elevating high density cholesterol, comprising administration of an effective amount of a phosphonate derivative of the formula according to claim 1.

41. A method for preventing and/or treating atherosclerosis, comprising administration of an effective amount of a phosphonate derivative of the formula according to claim 1.

42. A method for preventing and/or treating macular degeneration and retinitis pigmentosa comprising, administration of an effective amount of a phosphonate derivative of the formula according to claim 1.

43. A method for the preventing and/or treating stroke, comprising administration of an effective amount of a phosphonate derivative of the formula according to claim 1.

44. A method for the prevention of degenerative neuropathy, comprising administration of an effective amount of a phosphonate derivative of the formula according to claim 1.

45. The method of claim 44, wherein said degenerative neuropathy is associated with diabetic neuropathy or multiple sclerosis.

46. A method for the prevention and/or treatment of Alzheimer's disease or dementia comprising administration to a patient an effective amount of a phosphonate derivative of the formula according to claim 1.

47. The method of claim 46, wherein said patient is heterozygous or homozygous for apoE2 and/or apoE3 and wherein said phosphonate derivative increases apoE levels in said patient.

48. The method of claim 46, wherein said patient is heterozygous or homozygous for apoE4 and said phosphonate derivative decreases apoE levels in said patient.
